(19) United States
(12) Patent Application Publication
(10) Pub. No.: US 2015/0141257 A1
(43) Pub. Date: May 21, 2015

(54) SEQUENCE CAPTURE METHOD USING SPECIALIZED CAPTURE PROBES (HEATSEQ)

(71) Applicant: Roche NimbleGen, Inc., Madison, WI (US)

(72) Inventors: Thomas Albert, Verona, WI (US); Michael Brockman, Madison, WI (US); Daniel Lee Burgess, Madison, WI (US); Victor Lyamichev, Madison, WI (US); Jason Norton, Sun Prairie, WI (US); Jigar Patel, Madison, WI (US)

(21) Appl. No.: 14/338,921

(22) Filed: Jul. 23, 2014

Related U.S. Application Data
(60) Provisional application No. 61/861,695, filed on Aug. 2, 2013.

(51) Int. Cl. C12Q 1/68

(52) U.S. Cl. CPC C12Q 1/6876; C12Q 1/6874
USPC 506/2; 506/16; 506/26

(57) ABSTRACT

The present invention is a novel protocol for the massively parallel production of improved MIPs. The molecular improvements to the MIP cover the manufacturing of the probes, the workflow, the addition of unique sequence elements which connote sample specificity, and a sequence tag which uniquely identifies a specific molecule present in the initial sample population. Lastly, this invention also is combined with an empirical optimization strategy that overcomes issues of both locus representation and allelic bias. This improved technique is scalable and can be utilized to amplify targets comprised of a single locus' amplicon up to targeting more than 1 million loci.

### BACKGROUND OF THE DISCLOSURE

This invention relates to the field of methods for capture of targeted regions of a genome or complex DNA sample to enable efficient testing and/or detection of genetic polymorphisms found within the targeted region(s). Methods that efficiently capture targeted regions of a genome can enable the rapid sequencing-mediated discovery and detection of genetic polymorphisms associated with disease or other traits. Currently, hybridization based techniques that utilize double-stranded adapter-ligated sequencing libraries as inputs for target capture are time consuming and resource intensive. A traditional molecular inversion probe (MIP) based approach to target capture may reduce the workflow time prior to sequencing but is limited due to locus amplification/representation bias, allelic bias and systematic artifacts linked to specific sequencing platforms.

### BRIEF SUMMARY OF THE DISCLOSURE

The present invention is a novel protocol for the massively parallel production of improved MIPs. The molecular improvements to the MIP cover the manufacturing of the probes, the workflow, the addition of unique sequence elements which connote sample specificity, and a sequence tag which uniquely identifies a specific molecule present in the initial sample population. Lastly, this invention also is combined with an empirical optimization strategy that overcomes issues of both locus representation and allelic bias. This improved technique is scalable and can be utilized to amplify targets comprised of a single locus' amplicon up to targeting more than 1 million loci.

### BRIEF DESCRIPTION OF THE FIGURES

The features of this disclosure, and the manner of attaining them, will become more apparent and the disclosure itself will be better understood by reference to the following description of embodiments of the disclosure taken in conjunction with the accompanying drawing.

FIG. 1 are schematics describing the MIP precursor, the MIP precursor being amplified, and the restriction digestion of the amplified product.

FIG. 2 is an agarose gel purification of the enzyme digest product.

FIG. 3 depicts a 70-mer MIP probe hybridizing to a targeted strand of genomic DNA, and the extension/ligation of the MIP probe.

FIG. 4 is a gel purification of the MIP probes after extension/ligation (i.e., with “captured” product).

FIG. 5 is a graph showing the melting point ranges of probes with 20-mer target regions and the melting point ranges of probes with variable-length target regions (Tm balanced).

FIG. 6 is a graph showing the sequence coverage of fixed-length probes (inset) and Tm-balanced variable-length probes (main graph).

FIG. 7 are schematics describing the MIP precursor with UID, the amplification of the MIP precursor, the nicking of the amplified product, and the blocking oligonucleotide used during sequence capture.

FIG. 8 depicts hybridization of a MIP probe with UID sequence to a DNA target, and circularization of the MIP probe.

FIG. 9 shows a gel purification of the of the MIP probes after extension/ligation.

FIG. 10 depicts the use of the UID sequences.

FIG. 11 is a schematic depicting the synthesis of the MIP probes.

FIGS. 12 (12A and 12B) is a depiction of the workflow using the MIP probes.

FIG. 13 depicts the use of the sample index (MID) to identify the sample source.

FIG. 14 depicts the use of the UID sequences for event counting.

FIG. 15 shows the distribution of UID tags from one probe.

FIG. 16 demonstrates the results of probe rebalancing.

Although the drawings represent embodiments of the present disclosure, the drawings are not necessarily to scale and certain features may be exaggerated in order to better illustrate and explain the present disclosure. The exemplifications set out herein illustrate an exemplary embodiment of the disclosure, in one form, and such exemplifications are not to be construed as limiting the scope of the disclosure in any manner.

### DETAILED DESCRIPTION OF THE DISCLOSURE

Traditionally, Molecular Inversion Probes (MIPs) were single stranded nucleic acid probes having regions at or near their termini that were specifically complementary to two separate portions of a single stranded target nucleotide sequence. The probes “inverted” because they essentially took a circular configuration in order for the terminal target-specific portions to properly align and complement the target sequence, or conversely, that the target “inverted” in order to allow the same interaction between target regions and target-specific portions. The present invention provides improvements to MIPs by providing useful sequences for analysing data, improved synthesis methods for making such MIPs, and useful methods for optimizing the MIP probe pools.

The present invention includes a set of nucleic acid capture probes for reducing the complexity of a nucleic acid sample wherein each probe in the set contains a first terminal sequence that specifically hybridizes to a first target sequence present in the complex sample; a second terminal sequence that specifically hybridizes to a second target sequence present in the complex sample wherein the first and second target sequences are both located on the same target strand; and a linker sequence connecting the first terminal sequence and the second terminal sequence, the linker sequence containing a Unique Identifier (UID) sequence, wherein the UID is a randomly-generated tag sequence generated for each individual probe in the set of probes by random nucleotide synthesis during formation of the probes.

The present invention includes MIP probes with improved characteristics for determining allelic bias, locus amplification/representation bias, and systematic artifacts linked to specific sequencing platforms. Further, the invention also comprises certain methods of manufacturing such improved MIP probes using an array as the template for manufacturing the MIP probes. In some embodiments, the MIP probes are manufactured using an array as the template for the MIP probes. In certain embodiments, the invention comprises manufacturing the MIP probes with Maskless Array Synthesis (MAS) (see Singh-Gasson et al., Nature Biotechnology, 17: 974-978, 1999, hereby incorporated by reference).

In some embodiments, the MIP probes are designed using methods for optimizing probe design. In certain embodiments, the probe pools are designed using probe redistribution. Probe redistribution is performed by increasing or decreasing the relative concentration of particular probes during synthesis by synthesizing multiple replicates of the same probe over the surface of the array. In some embodiments, the probes in the probe pools are designed using probe length optimization. In some embodiments, the probes are designed using probe kinetic optimization, for example using Tm (melting temperature) to determine optimal probe design.

In some embodiments, the MIP probes contain a Molecular ID tag (MID). Such MIDs are essentially “bar code” nucleic acid sequences used for the purpose of identifying the sample from which the captured nucleic acid derives. Thus, the MID sequence allows for identification of the original sample through use of a sample specific identifier in which each of the captured sequences from a particular sample share a common barcode sequence. The MID sequence can be added to the sample in a number of different ways, including ligation with an adaptor sequence that contains the MID sequence, or through amplification using a primer containing the MID sequence.

In certain embodiments, the MID barcode is not present in the MIP probe until after the probe has been replicated and extended using a primer containing a primer site and a separate site containing the MID barcode. In some embodiments, the MID barcode is not added until after the MIP probe has contacted the target sequence. An example of this embodiment occurs when the MIP probe (without MID barcode) contacts its target sequence and specifically hybridizes. Through extension and ligation the MIP probe is circularized, then the circularized MIP probe is replicated/amplified using a primer with the additional MID barcode sequence.

The present invention includes a set of nucleic acid capture probes for reducing the complexity of a nucleic acid sample wherein each probe in the set. The probes comprise a first terminal sequence that specifically hybridizes to a first target sequence present in the complex sample and a second terminal sequence that specifically hybridizes to a second target sequence present in the complex sample. In this embodiment, the first and second target sequences are both located on the same target strand. The probes also have a linker sequence connecting the first terminal sequence and the second terminal sequence, the linker sequence comprising a Unique Identifier (UID) sequence. The UID is a randomly-generated tag sequence generated for each individual probe in the set of probes by chemically-derived random nucleotide synthesis during formation of the probes.

In certain embodiments, the probes further comprise a MID barcode wherein the probes used for a particular nucleic acid sample all contain the same MID barcode sequence. In this way, all results from a particular sample can be tracked.

Certain embodiments of the present invention also involve a method comprising a) synthesizing MIP precursors on an array wherein the precursors comprise one or more primer, one or more restriction site, and a first terminal target sequence near one end of the MIP precursor and a second terminal target sequence near the opposite end; b) amplifying the MIP precursors into solution; c) collecting the solution; and d) digesting the amplified precursors using one or more restriction enzymes to form MIP probes. In certain embodiments, the MIP precursor further comprises a Unique Identifier (UID) sequence.

Certain embodiments of the present invention also involve a method wherein the length of the first and/or second terminal target sequence is varied in order to closely approximate or match the melting temperatures of the two target sequences. This matching of melting point temperatures increases the sequence coverage for the MIP probe pools.

In one embodiment, the hybridizing step is performed in the presence of a blocking oligonucleotide designed to prevent the MIP probe from re-hybridizing to elements of the MIP precursors or amplification products thereof.

The MIP probes generated from the MIP precursor using the nicking enzymes (or other useful enzymes for this process, such as enzymes that can create a strand break, e.g., UDG/UNG) are used for targeted capture of regions defined by regions X and Y. The MIPs are nicked but double stranded, such that when denatured during the hybridization step, will release the active single stranded MIP from the double stranded MIP. In order to prevent this single stranded active MIP from re-hybridizing back to its complement forming back the original double stranded MIP, a 30-mer blocking oligo (300-24-1) is added. This oligo (300-24-1) since added in higher molar excess, will preferentially hybridize to the double stranded MIP cassette, preventing the previously release active single-stranded MIP to form a duplex. The active single-stranded MIPs are now available for targeted capture in subsequent extension+ligation reaction that would yield a circular MIP.

The present invention also includes embodiments wherein the MIP probes are used to identify portions of the target sequence by a) hybridizing the MIP probes to a nucleic acid sample; b) circularizing the MIP probes with a polymerase such that a portion of the nucleic acid sample is replicated and incorporated into the circularized MIP probes; c) substantially digesting linear nucleic acid using an exonuclease; and d) determining the sequence of the MIP probes. Once sequenced, the UID sequence (if used in the particular embodiment) can be used for determining if any UID sequence is over- or under-represented as compared to expected results.

In one embodiment of the methods of this invention, the array synthesis is performed using maskless array synthesis. MAS has the advantage of being an economical and highly flexible platform for nucleic acid synthesis and the use of MAS can therefore be advantageous over other synthetic methods.

In certain embodiments of the present invention, probe selection may require only one probe for coverage of a single exon, e.g., where the exon being targeted is small (usually less than 150 base pairs). In other embodiments, probe selection will require multiple probes to cover larger targets, such as larger exons, and the sequencing steps will be used to determine targeted overlaps and assemble the target sequence. In some embodiments, both large and small regions are targeted, requiring a mixture of both approaches.

In the present invention disclosure, certain terms have the meanings as ascribed in the following paragraphs.

The terms “a”, “an” and “the” generally include plural referents, unless the context clearly indicates otherwise.

The term “amplification” generally refers to the production of a plurality of nucleic acid molecules from a target nucleic acid wherein primers hybridize to specific sites on the target nucleic acid molecules in order to provide an inititation site for extension by a polymerase. Amplification can be carried out by any method generally known in the art, such as but not limited to: standard PCR, long PCR, hot start PCR, qPCR, RT-PCR and Isothermal Amplification. The term “amplifying” as used herein generally refers to the production of a plurality of nucleic acid molecules from a target nucleic acid wherein at least one primer hybridizes to specific site on the target nucleic acid molecules in order to provide an inititation site for extension by a polymerase. Amplification can be carried out by any method generally known in the art, such as but not limited to: standard PCR, long PCR, hot start PCR, qPCR, RT-PCR and Isothermal Amplification. Other amplification reactions comprise, among others, the Ligase Chain Reaction, Polymerase Ligase Chain Reaction, Gap-LCR, Repair Chain Reaction, 3SR, NASBA, Strand Displacement Amplification (SDA), Transcription Mediated Amplification (TMA), and Qb-amplification.

The term “complementary” generally refers to the ability to form favorable thermodynamic stability and specific pairing between the bases of two nucleotides at an appropriate temperature and ionic buffer conditions. This pairing is dependent on the hydrogen bonding properties of each nucleotide. The most fundamental examples of this are the hydrogen bond pairs between thymine/adenine and cytosine/guanine bases. In the present invention, primers for amplification of target nucleic acids can be both fully complementary over their entire length with a target nucleic acid molecule or “semi-complementary” wherein the primer contains additional, non-complementary sequence minimally capable or incapable of hybridization to the target nucleic acid.

The term “detecting” as used herein relates to a qualitative test aimed at assessing the presence or absence of a target nucleic acid in a sample.

The term “enriched” as used herein relates to any method of treating a sample comprising a target nucleic acid that allows to separate the target nucleic acid from at least a part of other material present in the sample. “Enrichment” can, thus, be understood as a production of a higher amount of target nucleic acid over other material.

The term “excess” generally refers to a larger quantity or concentration of a certain reagent or reagents as compared to another.

The term “hybridize” generally refers to the base-pairing between different nucleic acid molecules consistent with their nucleotide sequences. The terms hybridize“ and “anneal“ can be used interchangeably.

The terms “nucleic acid” or “polynucleotide” can be used interchangeably and refer to a polymer that can be corresponded to a ribose nucleic acid (RNA) or deoxyribose nucleic acid (DNA) polymer, or an analog thereof. This includes polymers of nucleotides such as RNA and DNA, as well as synthetic forms, modified (e.g., chemically or biochemically modified) forms thereof, and mixed polymers (e.g., including both RNA and DNA subunits). Exemplary modifications include methylation, substitution of one or more of the naturally occurring nucleotides with an analog, internucleotide modifications such as uncharged linkages (e.g., methyl phosphonates, phosphotriesters, phosphoamidates, carbamates, and the like), pendent moieties (e.g., polypeptides), intercalators (e.g., acridine, psoralen, and the like), chelators, alkylators, and modified linkages (e.g., alpha anomeric nucleic acids and the like). Also included are synthetic molecules that mimic polynucleotides in their ability to bind to a designated sequence via hydrogen bonding and other chemical interactions. Typically, the nucleotide monomers are linked via phosphodiester bonds, although synthetic forms of nucleic acids can comprise other linkages (e.g., peptide nucleic acids as described in Nielsen et al. (Science 254:1497-1500, 1991). A nucleic acid can be or can include, e.g., a chromosome or chromosomal segment, a vector (e.g., an expression vector), an expression cassette, a naked DNA or RNA polymer, the product of a polymerase chain reaction (PCR), an oligonucleotide, a probe, and a primer. A nucleic acid can be, e.g., single-stranded, double-stranded, or triple-stranded and is not limited to any particular length. Unless otherwise indicated, a particular nucleic acid sequence comprises or encodes complementary sequences, in addition to any sequence explicitly indicated.

The term “nucleotide” in addition to referring to the naturally occurring ribonucleotide or deoxyribonucleotide monomers, shall herein be understood to refer to related structural variants thereof, including derivatives and analogs, that are functionally equivalent with respect to the particular context in which the nucleotide is being used (e.g., hybridization to a complementary base), unless the context clearly indicates otherwise.

The term “oligonucleotide” refers to a nucleic acid that includes at least two nucleic acid monomer units (e.g., nucleotides). An oligonucleotide typically includes from about six to about 175 nucleic acid monomer units, more typically from about eight to about 100 nucleic acid monomer units, and still more typically from about 10 to about 50 nucleic acid monomer units (e.g., about 15, about 20, about 25, about 30, about 35, or more nucleic acid monomer units). The exact size of an oligonucleotide will depend on many factors, including the ultimate function or use of the oligonucleotide. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (Meth. Enzymol. 68:90-99, 1979); the phosphodiester method of Brown et al. (Meth. Enzymol. 68:109-151, 1979); the diethylphosphoramidite method of Beaucage et al. (Tetrahedron Lett. 22:1859-1862, 1981); the triester method of Matteucci et al. (J. Am. Chem. Soc. 103:3185-3191, 1981); automated synthesis methods; Maskless Array Synthesis as disclosed in Singh-Gasson et al., Nature Biotechnology, 17: 974-978, 1999, or the solid support method of U.S. Pat. No. 4,458,066, or other methods known to those skilled in the art.

The term “primer” refers to a polynucleotide capable of acting as a point of initiation of template-directed nucleic acid synthesis when placed under conditions in which polynucleotide extension is initiated (e.g., under conditions comprising the presence of requisite nucleoside triphosphates (as dictated by the template that is copied) and a polymerase in an appropriate buffer and at a suitable temperature or cycle(s) of temperatures (e.g., as in a polymerase chain reaction)). To further illustrate, primers can also be used in a variety of other oligonuceotide-mediated synthesis processes, including as initiators of de novo RNA synthesis and in vitro transcription-related processes (e.g., nucleic acid sequence-based amplification (NASBA), transcription mediated amplification (TMA), etc.). A primer is typically a single-stranded oligonucleotide (e.g., oligodeoxyribonucleotide). The appropriate length of a primer depends on the intended use of the primer but typically ranges from 6 to 40 nucleotides, more typically from 15 to 35 nucleotides. Short primer molecules generally require cooler temperatures to form sufficiently stable hybrid complexes with the template. A primer need not reflect the exact sequence of the template but must be sufficiently complementary to hybridize with a template for primer elongation to occur. In certain embodiments, the term “primer pair” means a set of primers including a 5′ sense primer (sometimes called “forward”) that hybridizes with the complement of the 5′ end of the nucleic acid sequence to be amplified and a 3′ antisense primer (sometimes called “reverse”) that hybridizes with the 3′ end of the sequence to be amplified (e.g., if the target sequence is expressed as RNA or is an RNA). A primer can be labeled, if desired, by incorporating a label detectable by spectroscopic, photochemical, biochemical, immunochemical, or chemical means. For example, useful labels include 32P, fluorescent dyes, electron-dense reagents, enzymes (as commonly used in ELISA assays), biotin, or haptens and proteins for which antisera or monoclonal antibodies are available.

In the sense of the invention, “purification”, “isolation” or “extraction” of nucleic acids relate to the following: Before nucleic acids may be analyzed in a diagnostic assay e.g. by amplification, they typically have to be purified, isolated or extracted from biological samples containing complex mixtures of different components. For the first steps, processes may be used which allow the enrichment of the nucleic acids. Such methods of enrichment are described herein.

The term “quantitating” as used herein relates to the determination of the amount or concentration of a target nucleic acid present in a sample.

“Target nucleic acid” is used herein to denote a nucleic acid in a sample which should be analyzed, i.e. the presence, non-presence, nucleic acid sequence and/or amount thereof in a sample should be determined. The target nucleic acid may be a genomic sequence, e.g. part of a specific gene, RNA, cDNA or any other form of nucleic acid sequence. In some embodiments, the target nucleic acid may be viral or microbial.

The terms “target nucleic acid”, and “target molecule” can be used interchangeably and refer to a nucleic acid molecule that is the subject of an amplification reaction that may optionally be interrogated by a sequencing reaction in order to derive its sequence information.

The terms “target specific region” or “region of interest” can be used interchangeably and refer to the region of a particular nucleic acid molecule that is of scientific interest. These regions typically have at least partially known sequences in order to design primers which flank the region or regions of interest for use in amplification reactions and thereby recover target nucleic acid amplicons containing these regions of interest.

The term “thermostable polymerase” refers to an enzyme that is stable to heat, is heat resistant, and retains sufficient activity to effect subsequent polynucleotide extension reactions and does not become irreversibly denatured (inactivated) when subjected to the elevated temperatures for the time necessary to effect denaturation of double-stranded nucleic acids. The heating conditions necessary for nucleic acid denaturation are well known in the art and are exemplified in, e.g., U.S. Pat. Nos. 4,683,202, 4,683,195, and 4,965,188. As used herein, a thermostable polymerase is suitable for use in a temperature cycling reaction such as the polymerase chain reaction (“PCR”). Irreversible denaturation for purposes herein refers to permanent and complete loss of enzymatic activity. For a thermostable polymerase, enzymatic activity refers to the catalysis of the combination of the nucleotides in the proper manner to form polynucleotide extension products that are complementary to a template nucleic acid strand. Thermostable DNA polymerases from thermophilic bacteria include, e.g., DNA polymerases from Thermotoga maritima, Thermus aquaticus, Thermus thermophilus, Thermus flavus, Thermus filiformis, Thermus species Sps17, Thermus species Z05, Thermus caldophilus, Bacillus caldotenax, Thermotoga neopolitana, and Thermosipho africanus.

The term “maskless array synthesis” (MAS) refers to light-directed synthesis of oligonucleotides on the surface of a substrate as an array in the absence of a physical mask, such as the method as described by Singh-Gasson et al., Nature Biotech, 17: 974-978 (October 1999), the teachings of which are hereby incorporated by reference. Briefly, the MAS technique generally uses a digital microarray mirror device (DMD) which consists of micromirrors to form virtual masks. These mirrors are individually addressable and can be used to create any given pattern or image in a broad range of wavelengths. The DMD forms an image on the surface of the substrate, wherein the substrate contains chemical moieties that are activated by light. A solution containing a given nucleotide is then washed over the surface of the substrate, and binds to the activated regions. The nucleotide in the solution contains are photoprotected with a protecting group that is photolabile. In a second round of synthesis, the DMD forms a second image onto selected regions of the substrate, thereby selectively activating the substrate in those regions, and a second given nucleotide (again, photoprotected) is washed over the substrate. This second nucleotide binds to those regions that have been activated during the second round of illumination. Thus, selected nucleotides can be added to selected regions, allowing for synthesis of an array of oligonucleotides through light-directed synthesis in the absence of a mask. This process is repeated numerous times in order to build the oligonucleotides sequences on a monomer-by-monomer basis.

Other methods of building arrays can also be used in the present invention, such as the use of chromium masks or spotting of oligonucleotides on an array. MAS provides improved flexibility and simplicity when used in the present invention, but other means of forming arrays are useful as well. Examples of the synthetic systems, besides MAS, that can be used in the present invention are those well-known methods used by Affymetrix, Oxford Gene Technologies, and Agilent.

The present invention involves synthesizing MIP precursor molecules on an array surface, then amplifying those MIP precursors into solution, where other manufacturing steps can then be performed. In certain embodiments, the MIP precursors are amplified through amplification systems such as PCR. In such embodiments, the MIP precursors are generally synthesized such that they contain primer sites useful for such later amplification steps.

In certain aspects of the invention, the probes are manufactured on the array so that they contain UID regions. UID regions are segments of the probes that are unique to the individual probe and the probe can be identified based upon the particular UID sequence present. UID sequences can be designed in several different ways, including pre-planning of the particular UID sequences to be used for the probes, random UID sequence generation via computer or other means followed by probe synthesis to incorporate the UID sequences into the probes, or through chemically-derived random synthesis. “Chemically-derived random synthesis” means that several of the nucleotides are mixed and simultaneously exposed to the synthesis surface during probe synthesis and allowed to randomly form into sequences with no pre-planning or prior random sequence determination. In one embodiment, a mixture of all four common nucleotides (A, C, T, G) useful for light-directed synthesis (e.g., masked array or maskless array synthesis) are mixed and added during several successive iterations of the synthesis and allowed to randomly bind to the light activated portions of the surface or array. In this embodiment, the order of the A, C, T or G will be random with no pre-planning of the sequence. Chemically-derived random synthesis provides the advantage of streamlining the probe production methods in that no steps are added to the workflow to pre-plan the sequence.

### EXAMPLES

### Example 1

### MIP Probe Pool Production and Purification

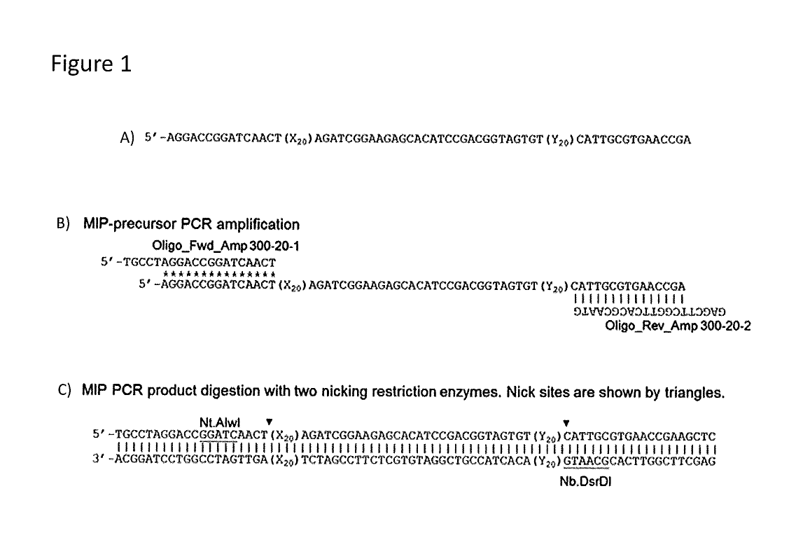

The protocol for conversion of MIP-precursors to MIPs is detailed in FIG. 1. FIG. 1A shows an example regarding a MIP-precursor molecule. In this example, the MIP precursor was formed by synthesis on a MAS unit such that the precursor was formed on an array surface. The MIP precursor molecule in this example contains two 15mer primer sites on the 5′ and 3′ termini. Adjacent to the terminal primer sites are two 20mer sites that are target specific regions, X₂₀ and Y20, which are complementary to particular sites that border a particular target region in the sample. Between X20 and Y20 is a linker region, in this case a 30mer sequence, which links the two target-specific sequences together.

The MIP precursor is then subjected to amplification using two primers, in this instance the primers are shown in FIG. 1B. There was both a forward and a reverse primer. The forward primer contains the same sequence as found on the 5′ terminal section of the MIP precursor molecule, while the reverse primer contains sequence complementary to the sequence at the 3′ terminal of the MIP precursor, as demonstrated in FIG. 1B. Thus, in the first amplification step, the reverse primer hybridizes to the MIP precursor and is extended, providing the complementary sequence to which the forward primer can bind in later amplification steps. In the present example, a chamber (Grace Bio-Lab, parts 05876702001 or 05871158001) having an inlet and outlet port was adhered to the MIP-precursor array, forming a chamber in which amplification was performed, using the MIP-precursor molecules as the amplification template. The amplification was performed in a thermal cycler, using a Slide Griddle Adaptor (BioRad, SGP0196). An in situ PCR master mix was prepared containing the following:

| Component | 1 Array |
|---|---|
| 10x ThermoPol Reaction Buffer | 110 μl |
| 25 mM dNTP | 5.5 μl |
| 50 μM Fwd Primer 300-20-1 | 20 μl |
| 50 μM Rev Primer 300-20-2 | 20 μl |
| 25 mM MgCl2 | 44 μl |
| H2O (PCR Grade) | 889.5 μl |
| Total Master Mix | 1089 μl |

The tube containing the master mix was placed in a 95° C. heat block for 5 minutes to de-gas. HotStartTaq enzyme was added (11 uL [5 U/ul]) to the mix and the amplification protocol started. In this example, the protocol used involved steps as follows: 1) heat array to 97° C./15 min, towards the end of which time 1 mL of PCR mix is loaded into the chamber, the loading port is sealed, any bubbles are removed and the second port is sealed; 2) the chamber is cycled 30 times through heat steps of 100° C./1 min; 48° C./1.5 min; 78° C./1 min; 3) the chamber is held at 72° C./15 min; and 4) the chamber is cooled to 4° C. as a final step.

After the amplification, one seal was removed and the liquid from the chamber removed and purified using Qiaquick PCR Purification kit (Qiagen) according to specifications. After purification, optical density measurements were used to determine concentration of the purified MIP-precursors. At this point in the process, the MIP precursors have been amplified and are in double stranded form as demonstrated in FIG. 1C.

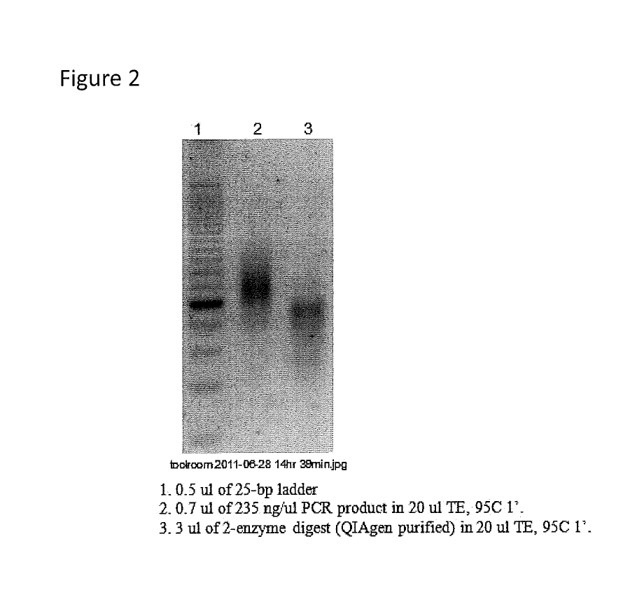

Further processing of the MIP precursors was performed. Specifically, the double stranded precursor molecules were further digested using two nicking restriction enzymes. Specifically, 5 μg (21.3 μl) of the PCR product was digested with 5 μl of Nt.Alw1 (10 U/μl, New England Biolabs) in 100 μl of 1× NeB2 at 37° C. for 3 hours. The product was run on a 2% agarose ethidium bromide gel. After this initial digest, the product was further digested with 5 μl of Nb.BsrD1 (10 U/μl, New England Biolabs) at 65° C. for 6 hours followed by 80° C. for 20 minutes. Incubation times can almost certainly vary, as can the enzymes used, concentrations, reactions conditions, etc. After digestion reactions were complete, the sample was purified with Qiagen nucleotide removal kit. Elution was performed using 30 μl of the standard elution buffer. DNA concentrations were determined (106 ng/μl), and samples run on 4% agarose gel, as shown in FIG. 2.

Lane 1 of the gel shown in FIG. 2 contains 0.5 μl of a 25 base pair ladder molecular weight standard. In lane 2, 0.7 μl of 235 ng/μl PCR product (i.e., the product after amplification but before restriction enzyme digestion) was run. Lane 3 shows the gel product when 3 μl of the 2-enzyme digest was run. Lane 3 therefore contains the final MIP probe pool used for hybridization to the sample.

### Example 2

### Use of the MIP Probe Pool for Capture of Targeted Regions

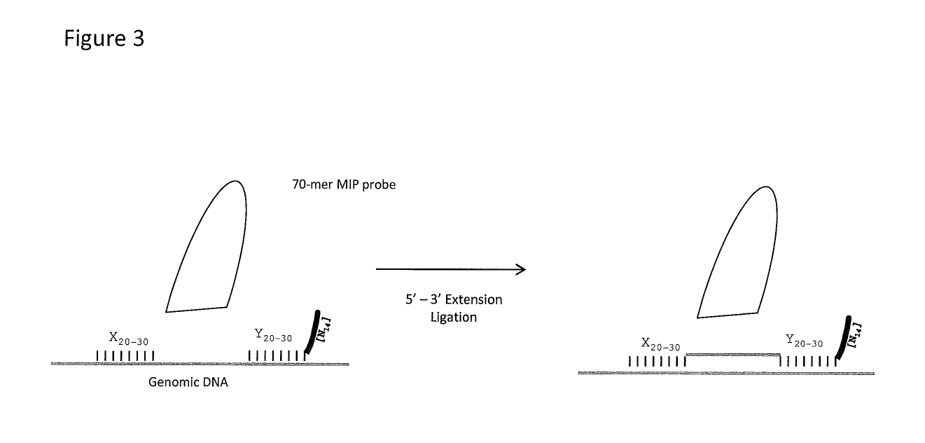

The protocol from Example 1 above results in 70-mer MIPs useful for hybridization to genomic DNA. For purposes of these examples, this pool was designated MIP480 mix. It is also readily recognized that such MIPs could be manufactured for use with other forms of nucleic acid targets, including cDNA, RNA, etc. Hybridization and extension steps wherein the MIP probes are contacting genomic DNA are depicted in FIG. 3.

In the present example, approximately 750 ng of hgDNA or 2.25×10⁵ copies of hgDNA were utilized. Keeping the MIP:genome equivalent ration to approximately 100:1, 1 pg of each probe (500 pg=0.5 ng of MIP480 mix) was used. These MIP calculations assume only 70 nucleotide MIP fragments are present. For the hybridization reaction, the following reagents were used:

| Reagent | Volume |
|---|---|
| 263 ng/μl Genomic DNA (female, Promega) | 3 μl 790 ng |
| 10X Ampligase buffer | 2.5 μl |
| 10 uM Blocking oligo 300-24-1 (300-20-3 in the first design) | 1 μl |
| 1 ng/μl MIP480 70-nt | 0.5 μl |
| Water to 25 μl | 18 μl |
| Mineral Oil | 30 μl |

As a control, replace gDNA with H₂O. Denature at 95° C. for 10 min, incubate at 60° C. for 36 h.

The captured DNA sequences (in this case, exons) were then circularized. A mix of 10 μl ligase and polymerase enzymes is prepared and added to each 25 μl capture reaction. The ligase/polymerase mix has the following reagents:

| Reagent | Volume | |
|---|---|---|
| 10X ampligase buffer | 1μl | (1X) |
| 5 U/μl ampligase | 1.75 μl | (0.25 U/μl) |
| 2 U/μl Phusion polymerase (NEB) | 0.7 μl | (0.04 U/μl) |
| 25 mM dNTP | 0.2 μl | (143 μM) |
| 100X NAD | 0.35 μl | (1X) |
| 5 M betain | 2.6 μl | (0.375 M) |
| Water | 3.4 μl | |

Add a total of 10 μl to the 25 μl capture reaction, incubate at 60° C. for 24 hours. The elongation/circularization step is depicted in FIG. 3.

A mixture of exonucleases was made with the following reagents (all from New England Biosciences):

| Reagent | Volume | |
|---|---|---|
| Exo I | 8.75 ul | (20 U/ul) |
| Exo III | 9 ul | (100 U/ul) |
| Exo T7 | 20 ul | (10 U/ul) |
| Exo T | 4 ul | (5 U/ul) |
| RecJf | 5 ul | (30 U/ul) |
| Lambda exo | 2 ul | (5 U/ul) |

To remove linear DNA, 2 ul of the exonuclease mix was added to each 35 ul ampligase reaction. The samples were incubated at 37° C. for 1 hour, 80° C. for 10 min, and 95° C. for 5 min.

After removal of the linear DNA, the remaining products were PCR amplified and purified in 25 ul reactions. For this PCR amplification (inverse PCR), the following reagents were used:

| Reagent | Volume | |
|---|---|---|
| 5X Phusion GC buffer | 5 μl | (1X) |
| 5 μM MIP PCR primer 300-24-2 | 2.5 μl | (500 nM) |
| 5 μM multiplex primer, Index 1 300-24-3 | 2.5 μl | (500 nM) |
| 10 mM dNTP (Promega) | 0.5 μl | (200 nM) |
| Sample (ext/lig/Exo circle) | 2.5 μl | |
| 2 U/μl Phusion Polymerase | 0.125 μl | (0.02 U/μl) |
| Water | 12.5 μl | |

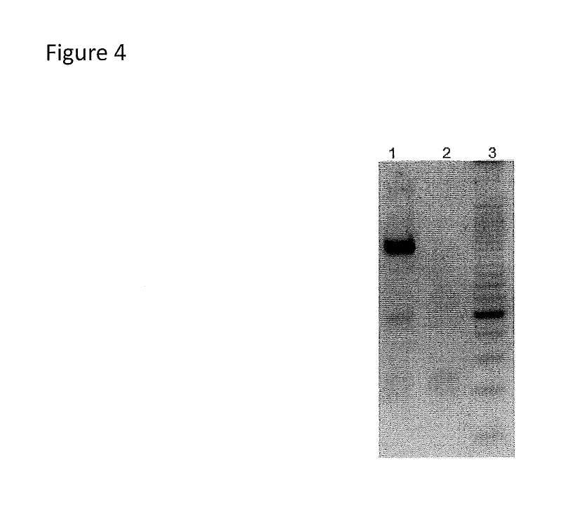

In this reaction, the multiplex primer contains the MID sequence for sample identification. For the PCR amplification, the reaction is held at 98° C. for 30 mins, then is cycled 30 times (98° C. for 10 mins/60° C. for 30 mins/72° C. for 1 min) and then is held at 72° C. for 2 min. PCR products were analysed in a 4% agarose gel (FIG. 4). In FIG. 4, lane 1 contains 5 ul of gDNA MIP capture PCR product in 20 ul of TE, lane 2 contains the control (water substituted for gDNA) and lane 3 contains 0.5 ul of a 25 base pair ladder. The DNA concentration from lane 1 was measured as 23.5 ng/ul or 130 nM. This amplified and purified product can then be used for sequencing, for example using Illumina TruSeq sequencing.

### Example 3

### MIP Protocol for Exon Capture Using 474 MIPs with Variable Length (Between 20-30 nt) for X and Y with Balanced Melting Temperature (Tm)

In this example, the MIP probes utilized have variable X and Y region lengths, between 20-30 nucleotides. In this embodiment, the Tm is calculated using standard formulas such that X and Y melting temperatures are nearly equivalent.

In the previous examples, the MIP probes were manufactured with fixed length 20-nt target specific regions, represented as such:

5′-(X₂₀) AGATCGGAAGAGCACATCCGACGGTAGTGT(Y₂₀), with X and Y representing the two 20 nucleotide long target-specific regions. In the present embodiment, the MIP probes have variable regions that can be represented as such:

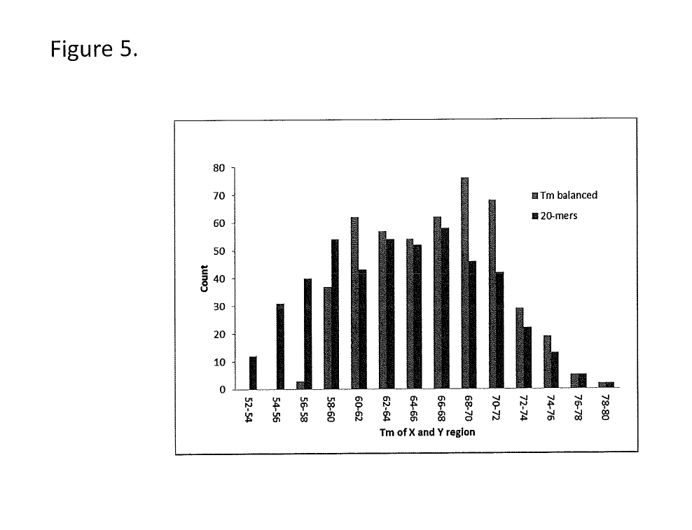

5′-(X₂₀₋₃₀) AGATCGGAAGAGCACATCCGACGGTAGTGT(Y₂₀₋₃₀), wherein the X region and the Y region do not necessarily have the same length. The Tm distribution of fixed length 20-nt probes and Tm balanced 20 to 30-nt probes is depicted in FIG. 5. In FIG. 5, the X-axis represents melting temperature of the probes while the Y axis represents the number of probes. As can be seen, varying the Tm of the probes concentrates the population into a smaller melting point range than when the X and Y region lengths are fixed. The table below contains the data used in FIG. 5:

| Fixed Length 20-mers | | Tm adjusted | |
|---|---|---|---|
| Count of Average Tm | | Count of Average Tm | |
| Average Tm | Total | Average Tm | Total |
| 52-54 | 12 | 52-54 | 0 |
| 54-56 | 31 | 54-56 | 0 |
| 56-58 | 40 | 56-58 | 3 |
| 58-60 | 54 | 58-60 | 37 |
| 60-62 | 43 | 60-62 | 62 |
| 62-64 | 54 | 62-64 | 57 |
| 64-66 | 52 | 64-66 | 54 |
| 66-68 | 58 | 66-68 | 62 |
| 68-70 | 46 | 68-70 | 76 |
| 70-72 | 42 | 70-72 | 68 |
| 72-74 | 22 | 72-74 | 29 |
| 74-76 | 13 | 74-76 | 19 |
| 76-78 | 5 | 76-78 | 5 |
| 78-80 | 2 | 78-80 | 2 |
| Grand Total | 474 | Grand Total | 474 |

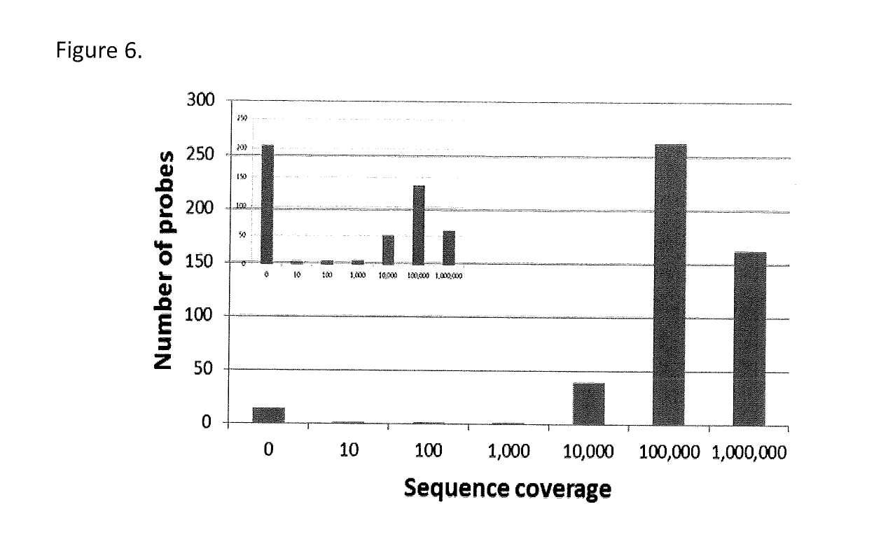

Experiments were run to determine the sequence coverage exhibited with the 20-nt fixed MIP probe pools versus the 20-30-nt variable MIP probe pools. Results of these experiments are seen in FIG. 6. FIG. 6 represents a frequency distribution of sequence coverage (no. of reads) comparing MIP probes designed with a fixed Tm (Inset) vs. Tm balanced design. Inset shows 45% of MIPs do not have any coverage (coverage of 0), whereas with Tm balanced design, the number of MIPs with no coverage drops to 3%, representing a ˜15 fold improvement in capture for the targeted regions represented by 474 MIPs. For the majority of MIPs in the Tm balanced design, the sequence coverage is relatively high, with reads upto a few million detected for some MIPs. In FIG. 6, the X-axis depicts the sequence coverage, which is a measure of the number of reads detected for this specific run on the IIlumina HiSeq for each MIP. Coverage is represented as a binned frequency distribution.

In that figure (see inset), fixed length MIP probe pools exhibited a large portion of the pool population that did not effectively exhibit any sequence coverage. In fact, 215/474 probes (45%) did not effectively cover the target sequence. In contrast, the main portion of the graph shows the sequence coverage when the Tm is balanced. As can be readily seen, the number of probes showing no sequence coverage dropped drastically, down to 15/474 (3%). Thus, embodiments wherein the Tm of the X and Y target regions is nearly equivalent confer an improvement over other embodiments wherein the X and Y regions are of set length.

### Example 4

### MIP Protocol for Exon Capture Using 474 MIPs with Variable Length Between 20-30 Nucleotides for X and Y Regions with Balanced Tm and N6 UID

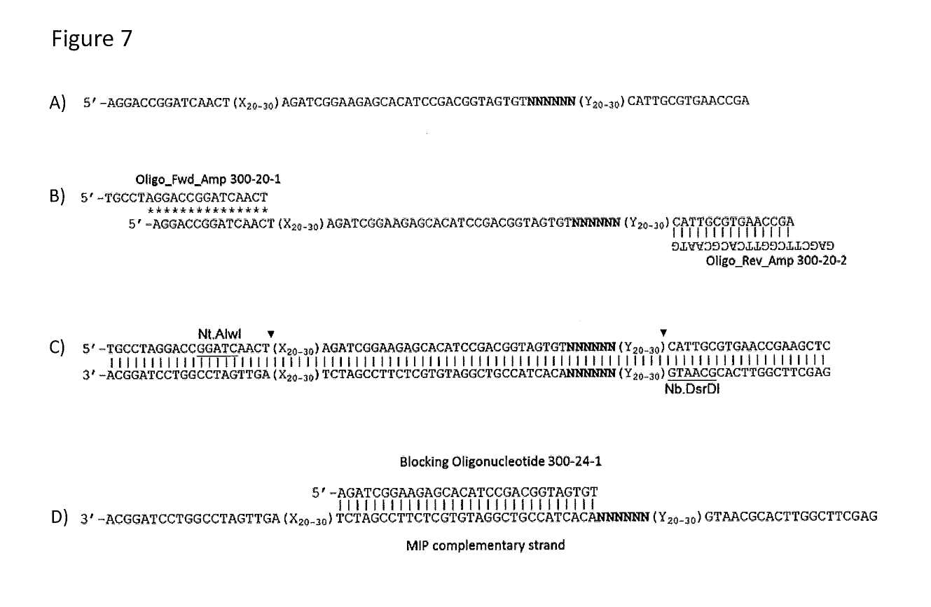

The general format for MIP precursors a UID sequence is depicted in FIG. 7A. In this example, the MIP probe has variable length target regions X and Y, connected with a linker region containing a UID region, denoted as NNNNNN (N6). the UID region can of course be synthesized with other strand lengths besides six nucleotides, and need only be long enough to derive the randomness needed for the particular experiment or use. This segment is a randomly-generated sequence that is synthesized in each probe (i.e., each probe has its own random UID sequence). This sequence can be used near the end of the sequencing workflow to determine if any particular probe target is being over-represented through amplification bias, locus amplification/representation bias, and systematic artifacts linked to specific sequencing platforms. In a similar workflow as described above, the MIP probes are synthesized, then amplified using primers (see FIG. 7B), then nicked with restriction enzymes and released as single stranded MIP pools (see FIG. 7C).

Single-stranded MIPs are hybridized to DNA (e.g., genomic DNA, but any nucleic acid molecules could be used). The complementary strand to the single-stranded MIPs are blocked using a blocking oligonucleotide, an example of which is depicted in FIG. 7D.

In this embodiment, MIP precursor templates were synthesized on an array using Maskless Array Synthesis (MAS). As in the example above, the MIP precursor array was adhered to a Grace Biolab Chamber and in situ PCR Master Mix was prepared. The in situ PCR Master Mix was substantially the same as in Example 1 above, except that the dNTP concentration was decreased to 10 mM and a larger volume (13.75 μl) was used in the Master Mix. The increased volume of the dNTP reagent was offset by a decrease in the volume of the forward and reverse primers (from 20 μl to 18 μl) and a decrease in the volume of water used. The tube containing the master mix was placed in a 95° C. heat block for 5 minutes to de-gas. HotStartTaq enzyme was added (11 uL [5 U/ul]) to the mix and the amplification protocol started. In this example, the protocol used involved steps as follows: 1) heat array to 97° C./15 min, towards the end of which time 1 mL of PCR mix is loaded into the chamber, the loading port is sealed, any bubbles are removed and the second port is sealed; 2) the chamber was cycled 15-18 times through heat steps of 100° C./1 min; 48° C./1.5 min; 78° C./1 min; 3) the chamber is held at 72° C. for 5 min; and 4) the chamber is cooled to 4° C. as a final step.

After the amplification, one seal was removed and the liquid from the chamber removed and purified using Qiaquick PCR Purification kit (Qiagen) according to specifications. After purification, optical density measurements were used to determine concentration of the purified MIP-precursors. Using 15 amplification cycles on one slide yielded 0.3 μg of MIP-precursors, while using 18 cycles on another slide yielded 2.3 μg. Additional amplification of the low amplified sample was performed in 1 ml PCR: 5×HF buffer (200 μl), 50 μM primer 300-20-1 (10 μl), 50 μM primer 300-22-2 (10 μl), 10 mM dNTP (20 μl), MIP precursor, 5 ng/μl (5 μl), water (750 μl), Phusion Polymerase (5 μl). The sample was heated to 98° C., then cycled 10 times (98° C. for 20 mins, 60° C. for 1 min, 72° C. for 1 min). PCR products were purified (Qiagen) in 50 μl H₂0. After this additional amplification, the DNA concentration was determined to be 117 ng/μl.

After amplification, the MIP precursors were treated with restriction enzymes: Digest 2.5 μg of PCR product with 5 μl of Nt.AlwI (10 u/μl, NEB) in 100 μl of 1×NEB2 at 37° C. for 3 h. Add 5 μl of Nb.BsrDI (10 u/μl, NEB). Incubate at 65° C. for 3 h followed by 80° C. for 20 min. Digestion reactions were purified with Qiagen nucleotide removal kit, and eluted in 30 μl elution buffer. DNA concentration was measured as 47 ng/μl, concentration of 86 nt Tm balanced N6 MIP was 47*86/(126+86)=19 ng/μl.

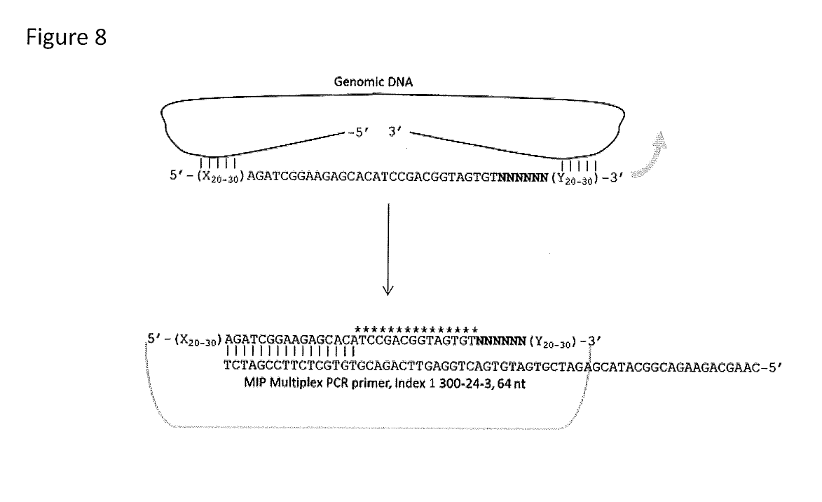

After the enzymatic treatment, the MIP probes are hybridized to genomic DNA, as illustrated in FIG. 8. For purposes of clarity, it should be noted that FIG. 8 depicts the genomic DNA in circularized fashion, as opposed to earlier figures which depict the MIP in circularized configuration. One of skill readily recognizes that conceptually either arrangement functions properly, and either configuration is only chosen because of particular preference for visualization.

In this example, the probes were hybridized to genomic DNA using the following reagents:

| Reagent | Volume |
|---|---|
| 263 ng/ul Genomic DNA (female, Promega) | 3 μl (790 ng) |
| 10X Ampligase buffer | 2.5 μl |
| 10 uM Blocking oligo 300-24-1 | 1 μl |
| 2 ng/ul MIP480 86-nt 400:1 ratio | 1 μl |
| Water to 25 ul | 17.5 μl |
| Mineral oil | 30 μl |

As a control, the gDNA was replaced with water. The samples were denatured at 95° C. for 10 min, and incubated at 61° C. for 36 hours.

In this embodiment, MIPs that were hybridized to genomic DNA were circularized by Ampligase after gap filling with Phusion polymerase. Ligase/polymerase mix were prepared with the following reagents:

| Reagent | Volume | |
|---|---|---|
| 10X ampligase buffer | 1 μl | (1X) |
| 5 U/μl ampligase | 1.75 μl | (0.25 U/μl) |
| 2 U/μl Phusion polymerase (NEB) | 0.7 μl | (0.04 U/μl) |
| 25 mM dNTP | 0.2 μl | (143 μM) |
| 100X NAD | 0.35 μl | (1X) |
| 5 M betain | 2.6 μl | (0.375 M) |
| Water | 3.4 μl | |

A total of 10 μl of the ligase/polymerase mix was added to each 25 μl capture reaction, and incubated at 60° C. for 24 hours.

To digest linear DNA, the samples were subjected to an exonuclease mix, consisting of the following reagents:

| Reagent | Conc. | Volume | Units |
|---|---|---|---|
| Exo I | 20 U/μl | 8.75 μl | 175 U |
| Exo III | 100 U/μl | 9 μl | 900 U |
| Exo T7 | 10 U/μl | 20 μl | 200 U |
| Exo T | 5 U/μl | 4 μl | 20 U |
| RecJf | 30 U/μl | 5 μl | 150 U |
| Lambda exo | 5 U/μl | 2 μl | 10 U |

To digest linear DNA, 2 μl of the exonuclease mix was added to each 35 μl Phusion/ampligase reaction. Samples were incubated at 37° C. for 1 hour, 80° C. for 10 min, 95° C. for 5 min.

The post-capture samples are then amplified and purified in 50 μl reactions:

| Reagent | Volume | |
|---|---|---|
| 5X Phusion GC buffer | 10 μl | (1X) |
| 5 uM MIP PCR primer 300-24-2 | 5 μl | (500 nM) |
| 5 uM MIP multiplex primer, Index 1, 300-24-3 | 5 μl | (500 nM) |
| 10 mM dNTP (Promega) | 1 μl | (200 nM) |
| Sample (ext/lig/Exo circle) | 5 μl | |
| H₂O | 25 μl | |
| 2 U/μl Phusion Polymerase | 0.25 μl | (0.02 U/μl) |

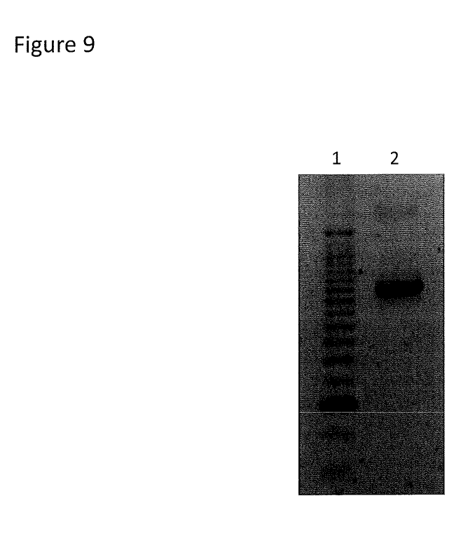

The samples were then amplified with thermal cycling: 98 C for 30 minutes, then 28 thermal cycles (98 C for 10 min/60 C for 30 min/72 C for 1 min). After amplification, 5 μl of the PCR products were analysed in 4% agarose gel, 30 min. The results are demonstrated in FIG. 9. Lane 1 shows a 25-bp ladder, lane 2 shows the PCR products.

The amplified samples were then sequenced on an Illumina sequencer.

### Example 5

### MIP Design for Exome Capture

In this example, the same protocol was used as described in Example 4 above, except that instead of synthesizing a pool of 474 MIP probes, the pool was increased to include 437,202 MIP probes (“437K pool”) with variable length between 20-30 nucleotides for the X and Y target regions with balanced Tm and N6 UID sequences on the individual probes.

Sequencing analysis was performed using the 437K pool to determine capture success rate. It was determined that the 437K pool has approximately an 82% capture success rate (i.e., 82% of the probes in the pool successfully capture targeted sequence).

### Example 6

### Use of UIDs

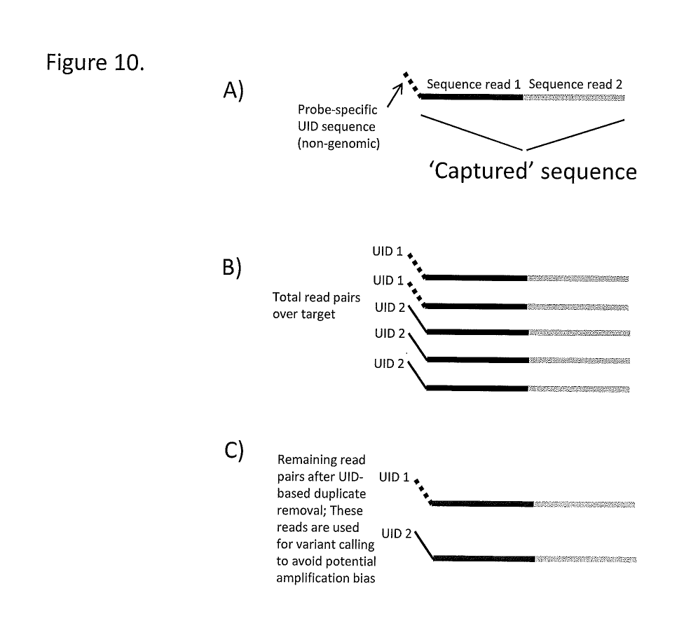

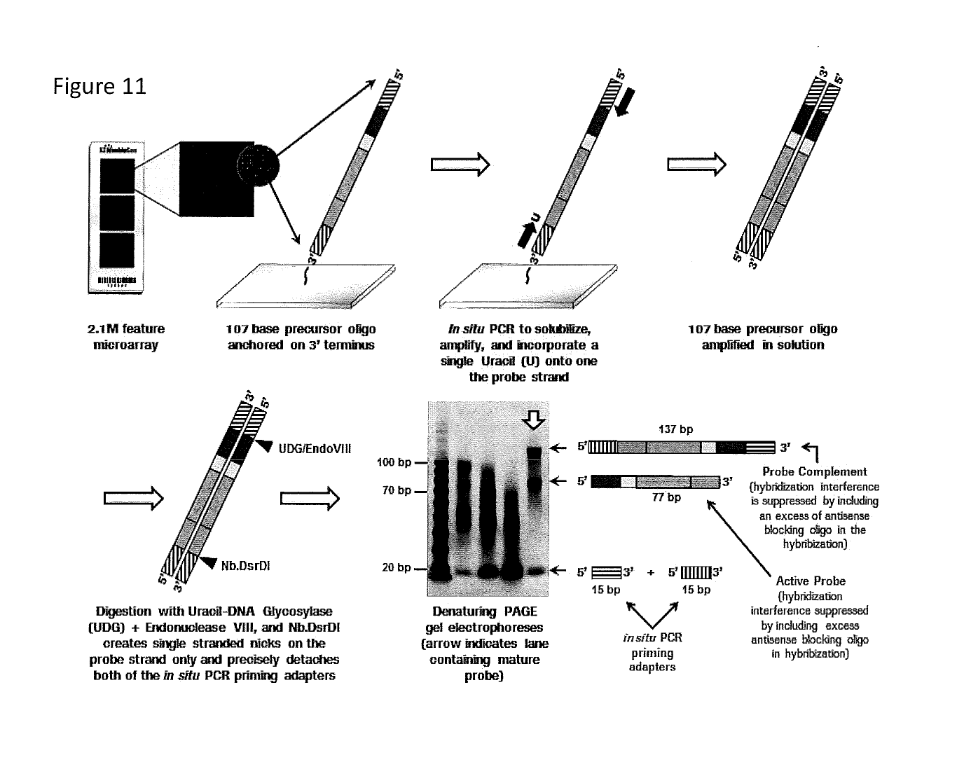

UIDs can be used to determine over- or under-representation of particular probes in the sequencing results, and are also useful for other purposes in which tracking the particular reads related to individual probes is important for data analysis. In one embodiment, UIDs are used to determine zygosity in the presence of potential allele bias introduced by amplification, as depicted in FIG. 10. For each MIP probe, sequencing reads will reveal the UID sequence that was synthesized for the probe (may appear in read 1, read 2, or both) and also contain the intended capture sequence (see FIG. 10A).

FIG. 10B shows that MIPs are primer based probes and so will produce a ‘stack’ of aligned sequence over the intended target. The probe-specific UID is used to distinguish molecular capture events. One UID may have multiple sequencing read pairs due to amplification. For the purpose of variant discovery, either a representative read pair or a consensus sequence is chosen from each set of read pairs containing an identical UID. If a capture event was amplified preferentially, the UID would have also been carried along. This UID-based duplicate read pair reduction removes that potential amplification bias (see FIG. 10C).

FIG. 11 exemplifies an embodiment of the manufacturing process of the MIP probes of the present invention. Using Maskless Array Synthesis, precursor molecules are synthesized on a monomer-by-monomer basis on an array, in this example a 2.1 M feature microarray. The precursor molecule may be anchored at the 3′ terminus to the surface of the array. Once synthesized, the array is subjected to in situ PCR to solubilize, amplify and incorporate a single uracil onto one probe strand. After amplification, the precursor is a double-stranded molecule in solution, containing the single uracil base. After amplification, the double-stranded molecule is subjected to digestion, in this example with Uracil-DNA glycosylase (UDG) and endonuclease VIII, and Nb.DSRDI creates single stranded nicks on the probe strand only, precisely detaching both of the in situ primer adapters. Denaturing PAGE gel electrophoresis demonstrates the formation of the probe and also shows the probe complement.

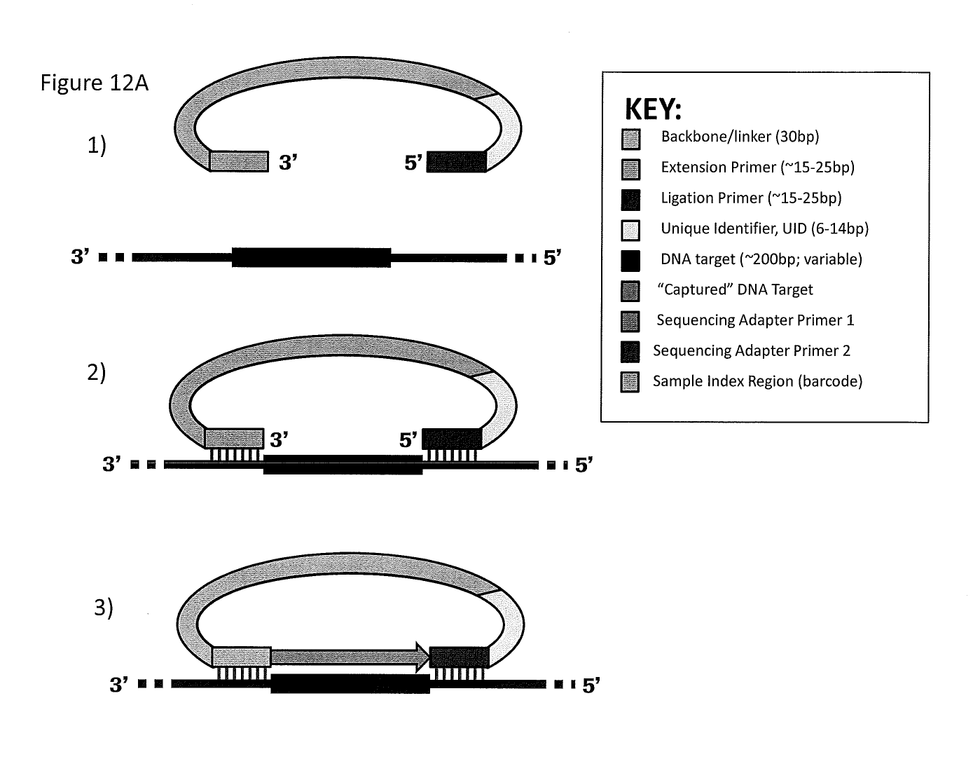

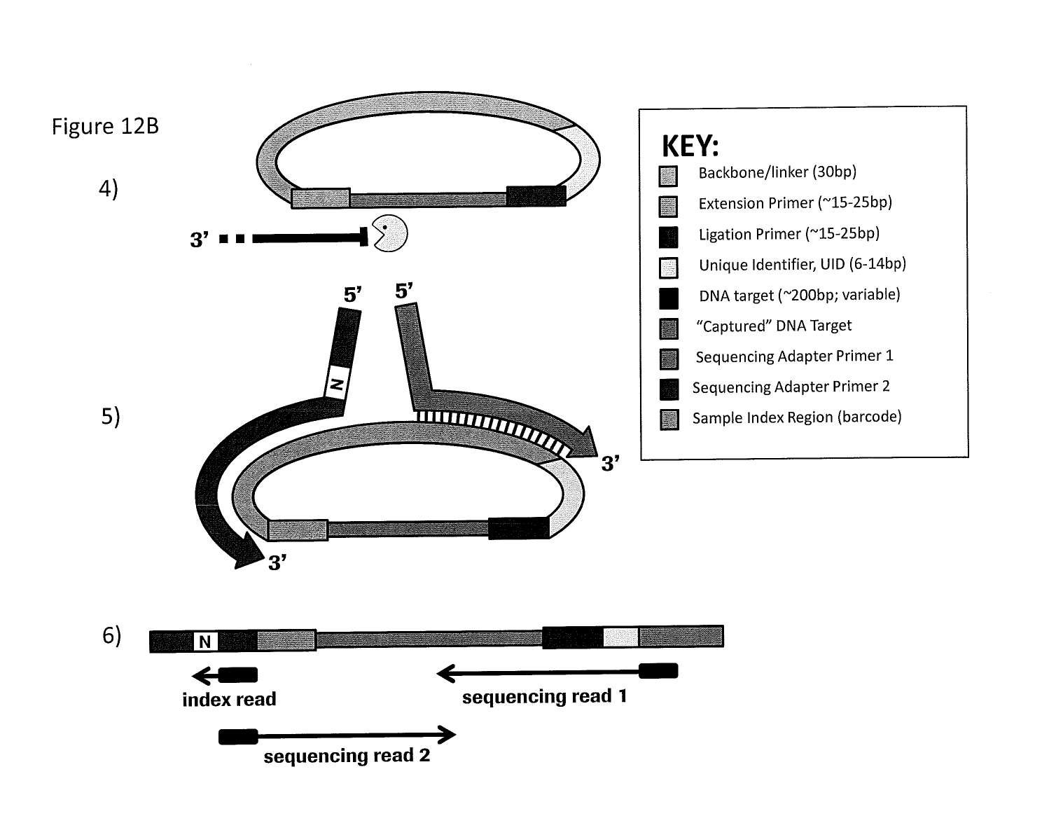

FIGS. 12A and 12B exemplify one embodiment of the workflow with respect to the MIP probes. In FIG. 12A1, the single-stranded MIP probes are mixed with target DNA in an appropriate ratio. The MIP probes and the target are allowed an appropriate amount of time to hybridize (FIG. 12A2), with the time being dependent on the complexity and ratio of the probe and the target. After hybridization, the MIP probe is extended and ligated to copy the target sequence and circularize the probe/target sequence (FIG. 12A3). Extension and ligation are accomplished using a mixture of DNA polymerase and DNA ligase.

After extension/ligation, single stranded template and probes are digested (FIG. 12B1). In some embodiments, a mixture of exonucleases such as ExoI and ExoIII are used for the digestion of the single-stranded molecules. Once the single stranded molecules are digested, the probe/target is amplified. In certain embodiments, sequencing adapters and sample index barcode (MID) sequences (denoted as “N” in FIG. 12B2) are incorporated. The MID code utilized a different sequence for each sample tested and allows for post amplification pooling before sequencing, as the sample can be identified by their MID code. FIG. 12B3 demonstrates the structure of the post-amplification, double-stranded product that is then ready for sequencing.

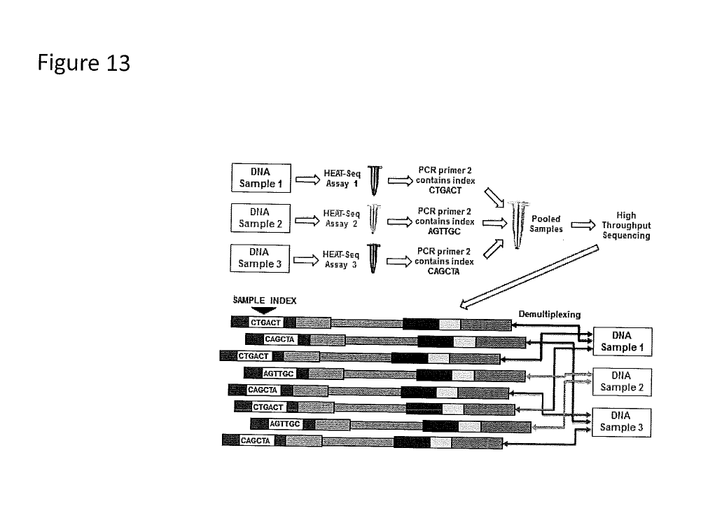

FIG. 13 exemplifies an embodiment of sample tracking using the present invention. The purpose of sample tracking is to allow captured, amplified DNA sequences from multiple experiments, each assaying a different genomic DNA sample, to be pooled prior to sequencing. This allows for more efficient matching of the vast amounts of sequencing data generated per sequencing run on a typical second generation instrument to the usually much lower sequence data requirements for analysis of captured sequences for any individual sample, thereby reducing costs, increasing efficiency, and permitting a higher sample throughput.

Sample tracking is accomplished by including a sample tracking index (usually a 6 to 14 nucleotide sequence) into one of the PCR primers used to amplify the circularized MIP probes. All amplicons of captured products originating from the same DNA sample will have the same tracking index, even though they are targeting many different regions within the genome of that DNA sample. After sequencing of the pooled captured products, the origin of each read-pair can be disambiguated by reading the associated index sequence.

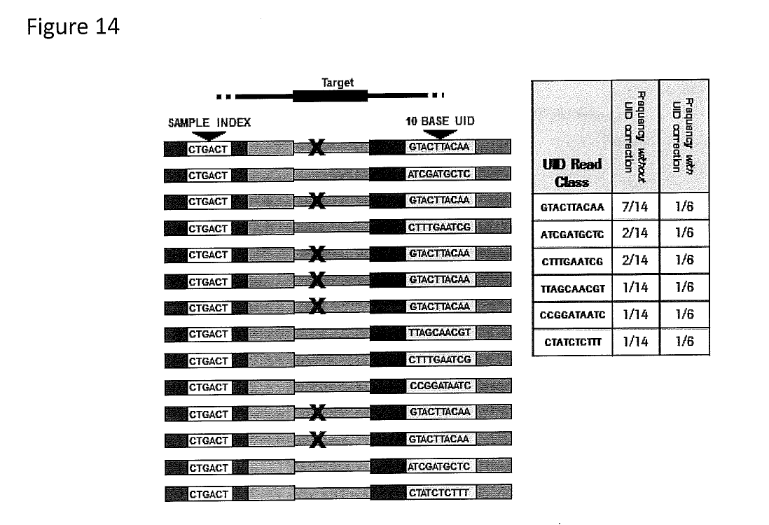

FIG. 14 exemplifies simulated data from an embodiment of event-counting using the UID sequences incorporated into the MIP probes. The purpose of event counting is to identify unique capture events for variant calling after removing the effects of amplification bias or other errors. The UID is a random sequence incorporated into every probe (not into the PCR primers themselves) and is copied upon amplification. Every probe molecule, even if it is used to target exactly the same exon in the same sample as another probe molecule, should have a different UID sequence. After sequencing, all read pairs that have the same UID sequence, except for one (the one with the highest sequence quality score) are discarded as likely PCR duplicates. All retained sequences are presumed to carry equal information value, and represent the true complexity of the sample. This capability is useful for determining the true frequency of a mutational event, such as a somatic mutation in a sample, or any variant in a mixed population. In FIG. 14, the simulated data from a single exon with and without UID correction is depicted. In the data without UID correction, the mutation (X) would be inaccurately measured at a frequency of 50% in the sample DNA due to biased amplification of the mutant allele. With UID correction, the actual frequency of the mutation in the sample DNA is revealed as 17%.

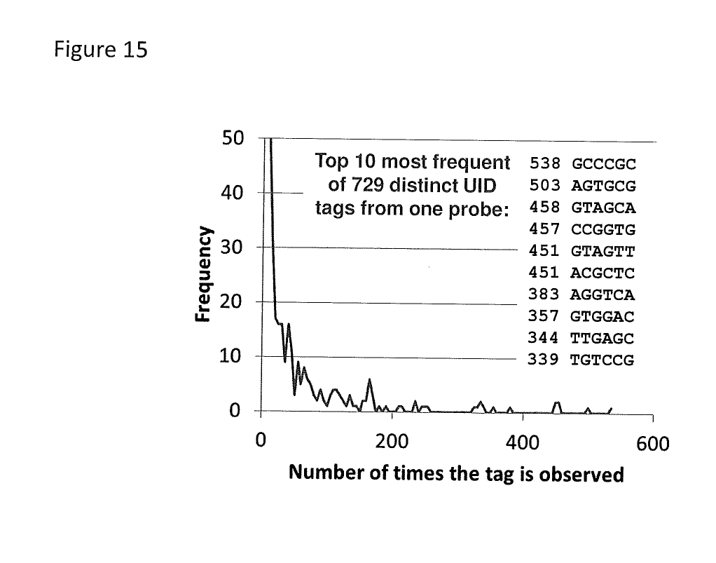

FIG. 15 shows the analysis of 23,517 read pairs corresponding to a single probe target (PTEN exon 4) within a larger MIP probe pool design. This analysis revealed 729 distinct 6-mer UID tags. The potential for strong amplification bias is demonstrated by the high (>300) frequency of some tags, while the UID facilitated elimination of the 96.4% of reads representing duplicate information.

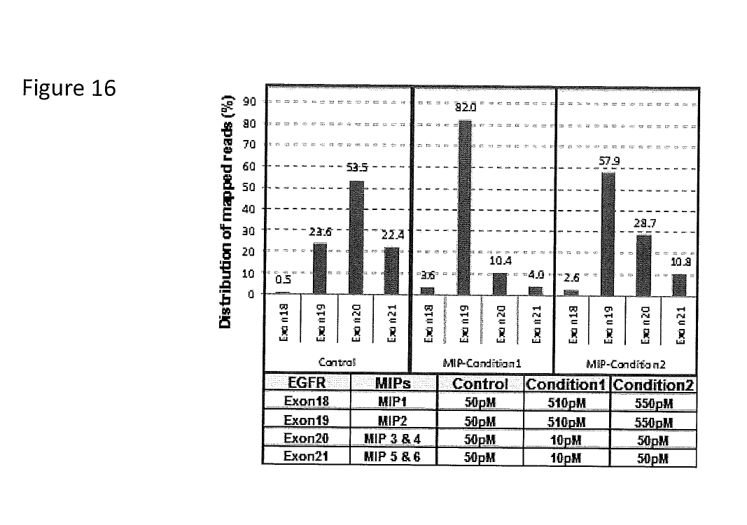

FIG. 16 shows the results of probe rebalancing. Four exons of the EGFR gene were targeted with 6 HEAT-Seq probes (obtained from IDT). 50 pM of probes were annealed to 500 ng gDNA and circularized over 4 hrs, then amplified. The probe/target constructs were then sequenced. 99% of the mapped reads were aligned to the targeted exons, with variable coverage depths of up to ˜100,000× (prior to UID deduplification). The highly variable sequence coverage depths obtained in the EGFR experiment exemplify a major inefficiency intrinsic to most highly-multiplexed, amplification-based, targeted sequencing methods. Rebalancing of probe ratios (right) can alter the sequence distribution among targets, but in unpredictable ways. Empirical and iterative approaches to probe design are currently the most effective solution (control=210,634 reads; MIP Condition1=429,202 reads; MIP Condition 2=313,346 reads).

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within the known or customary practice in the art to which this disclosure pertains.

All references cited in this specification are herewith incorporated by reference with respect to their entire disclosure content and the disclosure content specifically mentioned in this specification.

What is claimed is:

1. A set of nucleic acid capture probes for reducing the complexity of a nucleic acid sample wherein each probe in the set comprises:
a first terminal sequence that specifically hybridizes to a first target sequence present in the complex sample;
a second terminal sequence that specifically hybridizes to a second target sequence present in the complex sample wherein the first and second target sequences are both located on the same target strand; and
a linker sequence connecting the first terminal sequence and the second terminal sequence, the linker sequence comprising a Unique Identifier (UID) sequence, wherein the UID is a randomly-generated tag sequence generated for each individual probe in the set of probes by random nucleotide synthesis during formation of the probes.

2. The nucleic acid probes of claim 1 wherein the probes further comprise a MID barcode wherein the probes used for a particular nucleic acid sample all contain the same MID barcode sequence.

3. The nucleic acid probes of claim 1 wherein the UID sequence is generated through chemically-derived random synthesis.

4. The nucleic acid probes of claim 1 wherein the sequence length of the first terminal sequence and/or the second terminal sequence are of different lengths.

5. A method comprising
a) synthesizing MIP precursors on an array wherein the precursors comprise one or more primer, one or more restriction site, and a first terminal target sequence near one end of the MIP precursor and a second terminal target sequence near the opposite end;
b) amplifying the MIP precursors into solution;
c) collecting the solution; and
d) digesting the amplified precursors using one or more restriction enzymes to form MIP probes.

6. The method of claim 5, wherein the MIP precursor further comprises a Unique Identifier (UID) sequence.

7. The method of claim 5, further comprising
e) hybridizing the MIP probes to a nucleic acid sample; and
f) circularizing the MIP probes with a polymerase such that a portion of the nucleic acid sample is replicated and incorporated into the circularized MIP probes;
g) substantially digesting linear nucleic acid using exonucleases; and
h) determining the sequence of the MIP probes.

8. The method of claim 6, further comprising evaluating the sequence of the MIP probes and determining if any UID sequence is over- or under-represented as compared to expected results.

9. The method of claim 5 wherein the array synthesis is performed using maskless array synthesis.

10. The method of claim 5 wherein the length of the first and/or second terminal target sequence is varied in order to closely approximate the melting temperatures of the two target sequences.

11. The method of claim 7 wherein the hybridizing step is performed in the presence of a blocking oligonucleotide designed to prevent the MIP probe from re-hybridizing to elements of the MIP precursors or amplification products thereof.


SEQUENCE LISTING (17 sequences)

SEQ ID NO: 1 - DNA, 70 bp, Artificial Sequence
  Description of Artificial Sequence Synthetic oligonucleotide
nnnnnnnnnnnnnnnnnnnnagatcggaagagcacatccgacggtagtgtnnnnnnnnnnnnnnnnnnnn

SEQ ID NO: 2 - DNA, 90 bp, Artificial Sequence
  Description of Artificial Sequence Synthetic oligonucleotide
nnnnnnnnnnnnnnnnnnnnnnnnnnnnnnagatcggaagagcacatccgacggtagtgtnnnnnnnnnnnnnnnnnnnnnnnnnnnnnn

SEQ ID NO: 3 - DNA, 100 bp, Artificial Sequence
  Description of Artificial Sequence Synthetic polynucleotide
aggaccggatcaactnnnnnnnnnnnnnnnnnnnnagatcggaagagcacatccgacggtagtgtnnnnnnnnnnnnnnnnnnnncattgcgtgaaccga

SEQ ID NO: 4 - DNA, 20 bp, Artificial Sequence
  Description of Artificial Sequence Synthetic oligonucleotide
tgcctaggaccggatcaact

SEQ ID NO: 5 - DNA, 20 bp, Artificial Sequence
  Description of Artificial Sequence Synthetic oligonucleotide
gagcttcggttcacgcaatg

SEQ ID NO: 6 - DNA, 110 bp, Artificial Sequence
  Description of Artificial Sequence Synthetic polynucleotide
tgcctaggaccggatcaactnnnnnnnnnnnnnnnnnnnnagatcggaagagcacatccgacggtagtgtnnnnnnnnnnnnnnnnnnnncattgcgtgaaccgaagctc

SEQ ID NO: 7 - DNA, 126 bp, Artificial Sequence
  Description of Artificial Sequence Synthetic polynucleotide
aggaccggatcaactnnnnnnnnnnnnnnnnnnnnnnnnnnnnnnagatcggaagagcacatccgacggtagtgtnnnnnnnnnnnnnnnnnnnnnnnnnnnnnnnnnnnncattgcgtgaaccga

SEQ ID NO: 8 - DNA, 136 bp, Artificial Sequence
  Description of Artificial Sequence Synthetic polynucleotide
tgcctaggaccggatcaactnnnnnnnnnnnnnnnnnnnnnnnnnnnnnnagatcggaagagcacatccgacggtagtgtnnnnnnnnnnnnnnnnnnnnnnnnnnnnnnnnnnnncattgcgtgaaccgaagctc

SEQ ID NO: 9 - DNA, 30 bp, Artificial Sequence
  Description of Artificial Sequence Synthetic oligonucleotide
agatcggaagagcacatccgacggtagtgt

SEQ ID NO: 10 - DNA, 96 bp, Artificial Sequence
  Description of Artificial Sequence Synthetic oligonucleotide
nnnnnnnnnnnnnnnnnnnnnnnnnnnnnnagatcggaagagcacatccgacggtagtgtnnnnnnnnnnnnnnnnnnnnnnnnnnnnnnnnnnnn

SEQ ID NO: 11 - DNA, 64 bp, Artificial Sequence
  Description of Artificial Sequence Synthetic oligonucleotide
caagcagaagacggcatacgagatcgtgatgtgactggagttcagacgtgtgctcttccgatct

SEQ ID NO: 12 - DNA, 10 bp, Artificial Sequence
  Description of Artificial Sequence Synthetic oligonucleotide
gtacttacaa

SEQ ID NO: 13 - DNA, 10 bp, Artificial Sequence
  Description of Artificial Sequence Synthetic oligonucleotide
atcgatgctc

SEQ ID NO: 14 - DNA, 10 bp, Artificial Sequence
  Description of Artificial Sequence Synthetic oligonucleotide
ctttgaatcg

SEQ ID NO: 15 - DNA, 10 bp, Artificial Sequence
  Description of Artificial Sequence Synthetic oligonucleotide
ttagcaacgt

SEQ ID NO: 16 - DNA, 10 bp, Artificial Sequence
  Description of Artificial Sequence Synthetic oligonucleotide
ccggataatc

SEQ ID NO: 17 - DNA, 10 bp, Artificial Sequence
  Description of Artificial Sequence Synthetic oligonucleotide
ctatctcttt